(19) United States
(12) Patent Application Publication
(10) Pub. No.: US 2021/0338492 A1
(43) Pub. Date: Nov. 4, 2021

(54) REUSABLE DIAPER

(71) Applicant: HANDCRAFT LLC, New York, NY (US)

(72) Inventors: Joseph Mizrahi, Oakhurst, NJ (US); Marshall Mizrahi, Brooklyn, NY (US)

(21) Appl. No.: 16/862,085

(22) Filed: Apr. 29, 2020

(51) Int. Cl. A61F 13/49

(52) U.S. Cl. CPC A61F 13/49004; A61F 2013/49098; A61F 13/49011; A61F 13/49061

(57) ABSTRACT

A reusable diaper has an outer liquid resistant layer having a front waist portion, an elongated back waist portion and a crotch portion therebetween, an inner layer attached to the outer layer, and a plurality of first fasteners arranged along the front waist portion of the outer liquid resistant layer. The front waist portion is divided into a plurality of visually different sections each having at least one of the first fasteners thereon. Each section sandwiching a central section of the front waist portion can have a different color or surface pattern than that of the central section. Each section sandwiching that section and the central section therebetween has a still further different color or surface pattern. Each section corresponds to a predetermined diaper fitting size when one or more complementary second fasteners on ends of the elongated back waist portion engages a corresponding ones of the front waist portion first fasteners.

### BACKGROUND OF THE DISCLOSURE

This application relates to diapers for infants and children and more particularly to a reusable diaper that can be laundered and reused many times. Recently, reusable diapers have been developed that have plastic snap closures on opposite ends of the back portion to fasten the back portion to complementary sets of mating snaps or other fasteners on the front portion of the diaper.

In order to accommodate infants and children of different ages and hence size, there are typically up to ten sets of mating snaps on the front portion. In addition, to accommodate children of various sizes the diaper includes another set of several snaps arranged on the crotch portion of the diaper such that the diaper can be configured to cover a larger range of child sizes. The caregiver or parent putting the diaper on a squirming infant or child can have significant difficulty in matching the back snaps to the appropriate set of front portion snaps such that the fit is comfortable to the child yet snug around the child's leg so as to prevent excessive looseness of the crotch portion. Thus with all these fastener choices, the caregiver or parent can have some difficulty in choosing the appropriate set of snaps or other fasteners to properly fasten the diaper to the child. Therefore there is a need for a better way to facilitate matching appropriate fasteners for the child's size.

### SUMMARY OF THE DISCLOSURE

A reusable diaper in accordance with the present disclosure specifically addresses this need. One embodiment of a reusable diaper in accordance with the present disclosure has the front waist portion of the diaper divided into different visually coded sections, for example, by color or pattern. A central section of the front waist portion, for example is a first color, for example, red. Each section of the front waist portion adjacent to or sandwiching the central section is a second color, for example green. Each section of the front waist portion sandwiching the second color sections is a third color, such as yellow, and so on. Thus the care provider can easily adjust the fit of the diaper by engaging the back portion fasteners with corresponding color section fasteners on the front waist portion of the diaper thereby assuring that the leg openings created in the crotch portion by the fastened waist fasteners are appropriately sized and equal.

One embodiment of a reusable diaper in accordance with the present disclosure has an outer liquid resistant layer having a front waist portion, an elongated back waist portion and a crotch portion therebetween. Preferably the diaper has an inner layer attached to the outer layer, and there is a plurality of first fasteners arranged along the front waist portion of the outer liquid resistant layer. This front waist portion is divided into a plurality of visually different sections, arranged side by side, each having at least one of the first fasteners thereon. Each of the visually different sections corresponds to a predetermined waist size when one or more complementary second fasteners on the elongated back waist portion engage a corresponding one of the front waist portion first fasteners.

In one exemplary embodiment, each of the visually different sections has a surface pattern different from an adjacent surface pattern. In one embodiment a central section of the front waist portion of the diaper is a first color while each section sandwiching the central section is a second color. Similarly, each section sandwiching the sections having the second color has a third color different from the first and second colors and each section sandwiching the sections having the third color has a fourth color different from the first, second and third colors.

In one exemplary embodiment, the visually different sections differ in the pattern of each section spaced from a central section of the front waist portion of the diaper. For example, the central section may have no pattern. The sections on either side of the central section may have stripes, crosshatching or polka dots. Alternatively, each section can have printed thereon a different pattern.

In a still further alternative embodiment, each section may have a snap or other fastener of a different color rather than the fabric being a different color. In this case, for example, the central section may have one or more red fasteners thereon. The sections sandwiching the central section may have different color fastener or fasteners thereon, for example, green. The outer sections sandwiching the sections with green fasteners could have a further different color fastener thereon such as yellow fasteners, and so on.

A reusable diaper in accordance with the present disclosure may have as many as 8, 10 or twelve sets of fasteners across the width of the front waist portion of the diaper. Preferably the central section of the front waist portion has a set of 2 or 4 fasteners located thereon so that both ends of the rear portion may be fastened to the central section of the front waist portion. Each other section may have one or two fasteners thereon. For example, a reusable diaper for newborn infants preferably has two fasteners on its central section and a single fastener on each section adjacent the central section and those next sections sandwiching the central section therebetween.

Alternatively, a reusable diaper in accordance with the present disclosure has an outer liquid resistant layer having a front waist portion, an elongated back waist portion and a crotch portion therebetween, an inner layer attached to the outer layer, a detachable absorbent pad attached to the inner layer; and a plurality of first fasteners arranged along the front waist portion of the outer liquid resistant layer. The front waist portion is divided into a plurality of visually different sections each having at least one of the first fasteners thereon. A central section of the front waist portion of the outer liquid resistant layer has a first color. Each section sandwiching the central section has a second color different from the first color. Each section sandwiching the sections having the second color has a third color different from the first and second colors. Each section sandwiching the sections having the third color has a fourth color different from the first, second and third colors. In one embodiment, the central section of the front waist portion has a set of four first fasteners thereon and each other section of the front waist portion has a set of two first fasteners thereon. The back waist portion has opposing ends and each end of the back waist portion has a set of two second fasteners thereon configured to mate with the first fasteners on the front waist portion.

### DESCRIPTION OF THE DRAWINGS

FIG. 1 is a perspective front view of an embodiment of a diaper in accordance with the present disclosure.

FIG. 2 is a view as in FIG. 1 with the rear waist flap unfastened and behind the front waist flap extended to show the different front waist sections.

FIG. 3 is an open inside plan view of the diaper shown in FIGS. 1 and 2.

FIG. 4 is an open inside partially exploded view of the diaper shown in FIG. 3 with the removable absorbent pad insert separated from the inner layer of the diaper.

### DETAILED DESCRIPTION

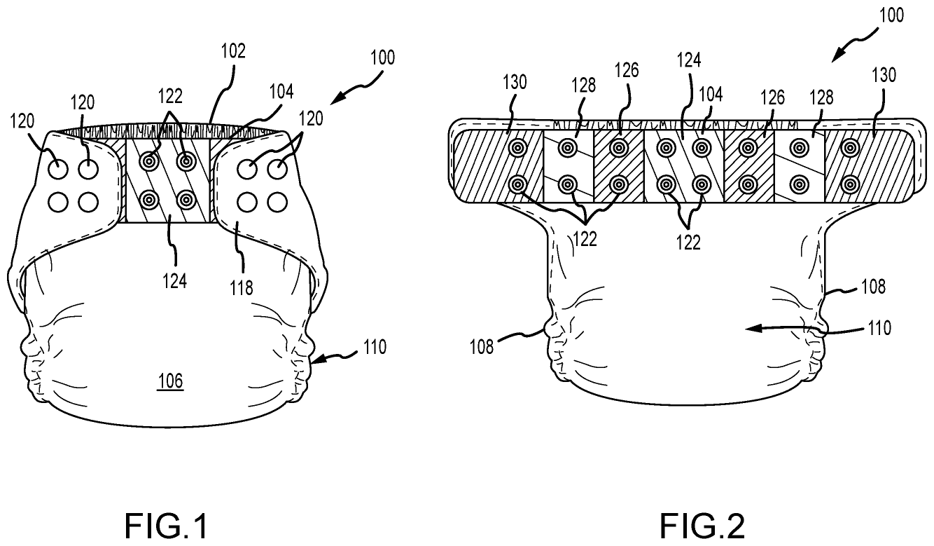

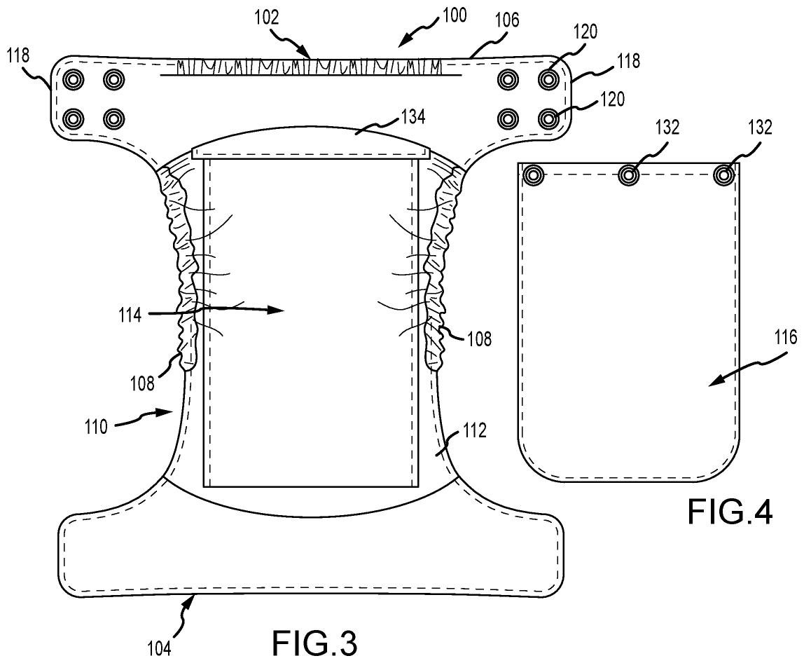

Turning now to the drawing, FIG. 1 is a front perspective view of one embodiment of a reusable diaper 100 in accordance with the present disclosure. FIG. 2 is the same view of diaper 100 except that the back waist portion 102 has been unfastened and the front waist portion 104 is stretched out straight in front of the back waist portion 102. FIG. 3 is an inside flattened plan view of the diaper 100.

The diaper 100 has an outer liquid resistant fabric layer 106. This outer layer 106 is formed from a piece of woven material such as a laminated polyester cloth. The outer fabric layer 106, best seen in FIG. 2, has a straight front waist portion 104, a straight back waist portion 102, and two concave side edges 108 forming sides of a crotch portion 110 extending between the front and back waist portions 104 and 102. Each of the side edges 108 hold elastic into place so as to wrap the crotch portion 110 around the child's leg when the diaper 100 is worn.

Sewn to the outer liquid resistant layer 106 is a matching shaped inner layer 112 of wicking fabric material such as stay dry suede cloth 100% polyester. The side edges 108 are preferably elasticized to fit around the wearer's legs. Similarly, the upper margin of the back waist portion may be elasticized so as to comfortably move with the wearer of the diaper 100. Preferably an inner, generally rectangular, padding layer 114 of, for example, a 2 layer bamboo terry cloth is sewn to the inner layer 112. Other suitable absorbent material or cloth may also be used for this inner padding layer 114.

Further, a removable absorbent pad 116 also of an absorbent material such as a bamboo terry cloth may be snap fastened to the inner layer 112 to provide additional absorptive material within the diaper 100.

The elongated back waist portion 102 of the diaper 100 has opposite ends 118. Each end 118 forms an elongated tab onto which a set of one, two or four female snaps 120 are installed. These snaps 120 engage corresponding male snaps 122 on the front waist portion of the diaper 100.

Looking now at FIGS. 1 and 2, the elongated front waist portion 104 is divided into 7 separate sections. The central section 124 carries 4 male snaps 122. There are two adjacent sections 126, one on each side of section 124 such that sections 126 sandwich section 124 therebetween. Sandwiching the sections 126 are two sections 128. Sandwiching the sections 128 are two end sections 130. Each section 124, 126, 128 and 130 has a different color. For example, the central section 124 may be blue. The adjacent sections 126 may be aqua. The next adjacent sections 128 may be forest green, and the last sections 130 may be lime green. Alternatively the colors may be more or less distinctive or subtle. For example, using pastels, the central section 124 may be an orange pastel, the adjacent sections 126 yellow pastel, the next adjacent sections 128 a light green pastel, and the last or end sections 130 blue. In FIG. 2, the different colors are indicated by textural shadings of grey. In alternative embodiments of the diaper 100, different shades or textures or prints could be used in each section so long as they are visually distinguishable. For example, the central section 124 could have a checkerboard design. The adjacent sections 126 could have vertical stripes. The next adjacent sections 128 could have horizontal stripes, and the last sections 130 could be plain fabric.

As is shown in FIG. 1 and FIG. 2, there is an array of snaps 122 arranged along the front waist portion of the diaper 100. In the illustrated embodiment the array is a 2×8 array, with the central section carrying one set of four snaps and each of the other sections carrying a set of 2 snaps each. Alternatively the array may be 1×6 or 1×8 for a newborn diaper size, accordingly with two in the central section and one each in the other sections. It is presently envisioned to have three sizes of diaper 100. A first size would be for a newborn baby, and would have an array of 1×6, with two in the central section and one each in the other sections. A second or mid size, would have a 2×6 array. A third size, for a large child, would perhaps have a 2×8 or 2×10 array on the front waist portion of the diaper 100.

The back waist portion 102 may have a set of one, two or four snaps 120 on each end 118 for mating with the snaps 122 arranged on the front waist portion 104 of the diaper 100. In FIG. 1 four snaps are shown. In a diaper 100 for a newborn, one snap 120 would be appropriate. The diaper 100 shown in FIGS. 1-4 is typical for diaper 100 sizes 2 and 3, thus sized for middle and large, e.g., for a child weighing 15-30 lbs, and for a child weighing 30-40 lbs.

The outer layer 106 is preferably sewn to the inner layer 112 as indicated by the dashed lines in FIGS. 3 and 4. The diaper 100 in accordance with the present disclosure preferably includes a reusable absorptive pad 116 that can be snapped via snaps 132 or otherwise fastened to the back waist portion 102 so as provide a flap that extends over the crotch portion 110 to the front waist portion 104. Optionally a flap 134 may be provided on the inner layer 112 to cover the snaps (not shown) on the inner layer 112 that mate with the snaps 132 on the insert 116. This flap 134 covers the snaps 132 so that they do not touch the child's skin in order to minimize any skin irritation.

There are many variations and alternatives that may be apparent to one skilled in the art upon reading this disclosure. For example, to enhance visible recognition, the sections could have both color and textural distinctions and could have color coded snaps as well. The embodiments described above are merely examples in accordance with the present disclosure. Accordingly all such variations and alternatives are encompassed herein as defined by the appended claims.

What is claimed is:

1. A reusable diaper comprising:
an outer liquid resistant layer having a front waist portion, an elongated back waist portion and a crotch portion therebetween;
an inner layer attached to the outer layer; and
a plurality of first fasteners arranged along the front waist portion of the outer liquid resistant layer, wherein the front waist portion is divided into a plurality of visually different sections each having at least one of the first fasteners thereon, wherein each section corresponds to a predetermined size when one or more complementary second fasteners on the elongated back waist portion engages a corresponding one of the front waist portion first fasteners.

2. The reusable diaper according to claim 1 wherein the plurality of visually different sections have different surface patterns.

3. The reusable diaper according to claim 1 wherein a central section of the front waist portion of the diaper has a first color.

4. The reusable diaper according to claim 2 wherein each section sandwiching the central section has a second color different from the first color.

5. The reusable diaper according to claim 3 wherein each section sandwiching the sections having the second color has a third color different from the first and second colors.

6. The reusable diaper according to claim 4 wherein each section sandwiching the sections having the third color has a fourth color different from the first, second and third colors.

7. The reusable diaper according to claim 1 wherein at least one of the first fasteners on a central one of the sections of the front waist portion of the diaper has a first color.

8. The reusable diaper according to claim 7 wherein at least one of the first fasteners on each section sandwiching the central section has a second color different from the first color.

9. The reusable diaper according to claim 8 wherein at least one of the first fasteners on each section sandwiching the sections having a fastener having the second color has a third color different from the first and second colors.

10. The reusable diaper according to claim 9 wherein at least one of the first fasteners on each section sandwiching the sections having a fastener having the third color has a fourth color different from the first, second and third colors.

11. A reusable diaper comprising:
an outer liquid resistant layer having a front waist portion, an elongated back waist portion and a crotch portion therebetween;
an inner layer attached to the outer layer;
a detachable absorbent pad attached to the inner layer; and
a plurality of first fasteners arranged along the front waist portion of the outer liquid resistant layer, wherein the front waist portion is divided into a plurality of visually different sections each having at least one of the first fasteners thereon, and wherein a central section of the front waist portion of the outer liquid resistant layer has a first color.

12. The reusable diaper according to claim 11 wherein each section sandwiching the central section has a second color different from the first color.

13. The reusable diaper according to claim 12 wherein each section sandwiching the sections having the second color has a third color different from the first and second colors.

14. The reusable diaper according to claim 13 wherein each section sandwiching the sections having the third color has a fourth color different from the first, second and third colors.

15. The reusable diaper according to claim 11 wherein the central section of the front waist portion has a set of four first fasteners thereon and each other section of the front waist portion has a set of two first fasteners thereon.

16. The reusable diaper according to claim 15 wherein the back waist portion has opposing ends and wherein each end has a set of two second fasteners thereon configured to mate with the first fasteners on the front waist portion.

17. The reusable diaper according to claim 16 wherein two or more of the sections of the front waist portion have different colors.

18. The reusable diaper according to claim 16 wherein the central section has a first color.

19. The reusable diaper according to claim 18 wherein the sections sandwiching the central section have a second color different from the first color.

20. The reusable diaper according to claim 19 wherein the sections sandwiching the sections sandwiching the central section have a third color different from the first and second colors.
